(19) United States
(12) Patent Application Publication
(10) Pub. No.: US 2003/0135271 A1
(43) Pub. Date: Jul. 17, 2003

(54) In-vivo adjustable intraocular lens

(76) Inventor: Mark H. Bandhauer, Orange, CA (US)

Correspondence Address: Bausch & Lomb Incorporated, One Bausch & Lomb Place, Rochester, NY, 14604-2701, US

(21) Appl. No.: 10/314,864

(22) Filed: Dec. 9, 2002

Related U.S. Application Data
(60) Provisional application No. 60/345,291, filed on Dec. 21, 2001.

(51) Int. Cl. A61F 2/16

(52) U.S. Cl. 623/6.22; 623/6.46

(57) ABSTRACT

An IOL having at least one, but preferably a plurality of frangible structures formed between the haptics and optic of the IOL wherein one or more of the frangible structures may be selectively severed in-vivo to adjust the position of the IOL in the eye which, in turn, adjusts the focal power of the IOL in-vivo in a controlled and predictable manner.

### BACKGROUND OF THE INVENTION

The present invention relates to intraocular lenses (IOLs) for implantation in an aphakic eye where the natural lens has been removed due to damage or disease (e.g., a cataractous lens). The present invention more particularly relates to a novel IOL designed to provide a selectively changeable optic power in-vivo in order to finely adjust a particular patient's optic correction immediately following implantation of the IOL.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary muscle which enables the eye to accommodate for near and far vision. The extracapular method further provides a natural centering and locating means for the IOL within the eye. The capsular bag also continues its function of providing a natural barrier between the aqueous humor at the front of the eye and the vitreous humor at the rear of the eye.

One difficulty when implanting an IOL into the lens capsule is being able to precisely predict how the IOL will stabilize in the capsule. Thus, even though the surgeon selects an IOL with the appropriate power correction for a given patient's prescriptive correction, once the IOL has been implanted into the capsule, it may unpredictably shift within the capsule resulting in a variation from the intended optical power correction of the IOL in-vivo. Unintended shifts in IOL positioning may be caused by a variety of factors, including changes in eye parameters due to the surgery, as well as simply being unable to precisely predict how the IOL will position itself in the capsular bag. Should unintended IOL shifting occur, the patient is usually left with the prospect of having to wear spectacles to compensate for the power variation. Understandably, it is preferable that the IOL power correction itself provide the patient with the correct power correction, without having to resort to spectacles to compensate for an unintended power variation.

In view of this problem, it would be desirable to have an IOL whose focal power is selectively changeable in-vivo through a simple IOL design and focal power adjustment technique.

### SUMMARY OF THE INVENTION

The present invention solves the deficiencies of the prior art by providing an IOL having at least one, but preferably a plurality of frangible structures formed between the haptics and optic of the IOL wherein one or more of the frangible structures may be selectively severed in-vivo to adjust the position of the IOL in the eye which, in turn, adjusts the focal power of the IOL in-vivo in a controlled and predictable manner.

### BRIEF DESCRIPTION OF THE DRAWING

FIG. 1 is a cross-sectional view of a human eye showing the natural lens within the capsular bag of the eye;

FIG. 2 is a cross-sectional view of a human eye showing the natural lens removed and replaced with an IOL;

FIG. 3 is a plan view of a preferred embodiment of the inventive IOL;

FIG. 4 is a side elevational view thereof;

FIGS. 5a-c are paired plan and side elevational views showing an IOL manufactured with pre-stress according to an embodiment of the present invention in various stages of implantation and adjustment; and

FIGS. 6a-c are paired plan and side elevational views showing an IOL manufactured without pre-stress according to another embodiment of the present invention in various stages of implantation and adjustment.

### DETAILED DESCRIPTION

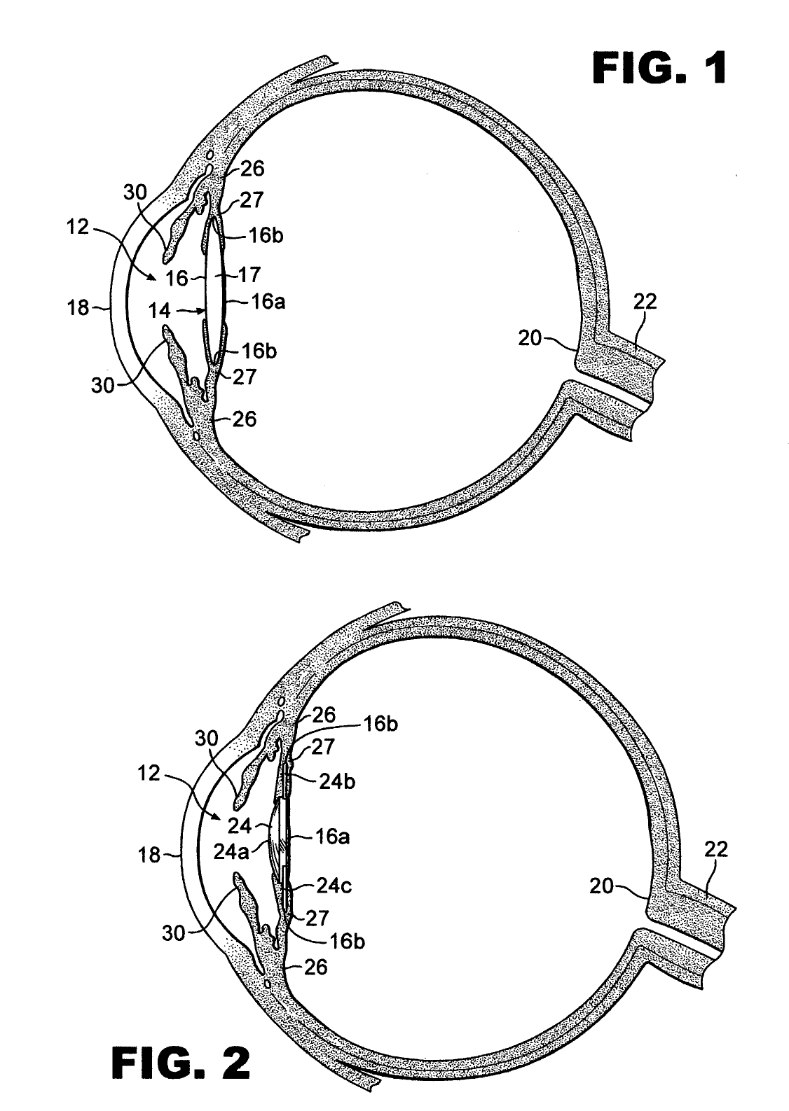

Referring now to the drawing, there is seen in FIG. 1 a cross-sectional view of a human eye 10 having an anterior chamber 12 and a posterior chamber 14 separated by the iris 30. Within the posterior chamber 14 is a capsule 16 which holds the eye's natural crystalline lens 17. Light enters the eye by passing through the cornea 18 to the crystalline lens 17 which act together to direct and focus the light upon the retina 20 located at the back of the eye. The retina connects to the optic nerve 22 which transmits the image received by the retina to the brain for interpretation of the image.

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIG. 2. Although there are many different IOL designs as well as many different options as to exact placement of an IOL within an eye, the present invention concerns itself with an IOL for implanting inside the substantially ovoid-shaped capsule 16 of eye 10. This implantation technique is commonly referred to in the art as the “in-the-bag” technique. In this surgical technique, a part of the anterior portion of the capsular bag is cut away (termed a “capsularhexis”) while leaving a remnant anterior portion and full posterior capsule 16a intact and still secured to the ciliary body 26.

Thus, in the “in-the-bag” technique of IOL surgery, the IOL is placed inside the capsule 16 which is located behind the iris 30 in the posterior chamber 14 of the eye. An IOL includes a central optic portion 24a which simulates the extracted natural lens by directing and focusing light upon the retina, and further includes means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the capsule (see FIG. 2).

As explained in the Background section hereof, an IOL may unpredictably shift within the eye during or after implantation which causes a shift in the originally intended IOL power correction due to a corresponding change in focal length. It has been found that an IOL optic positional shift as small as about 0.3 mm translates into a diopter shift of about 0.5 D. The present invention allows in-vivo adjustment of the position of the IOL to correct for unintended power correction shifts.

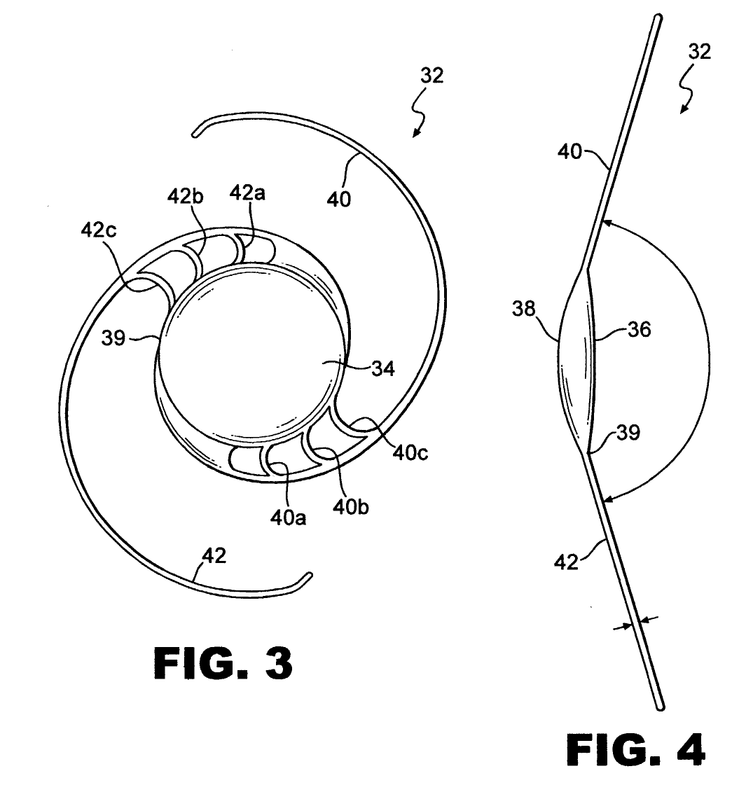

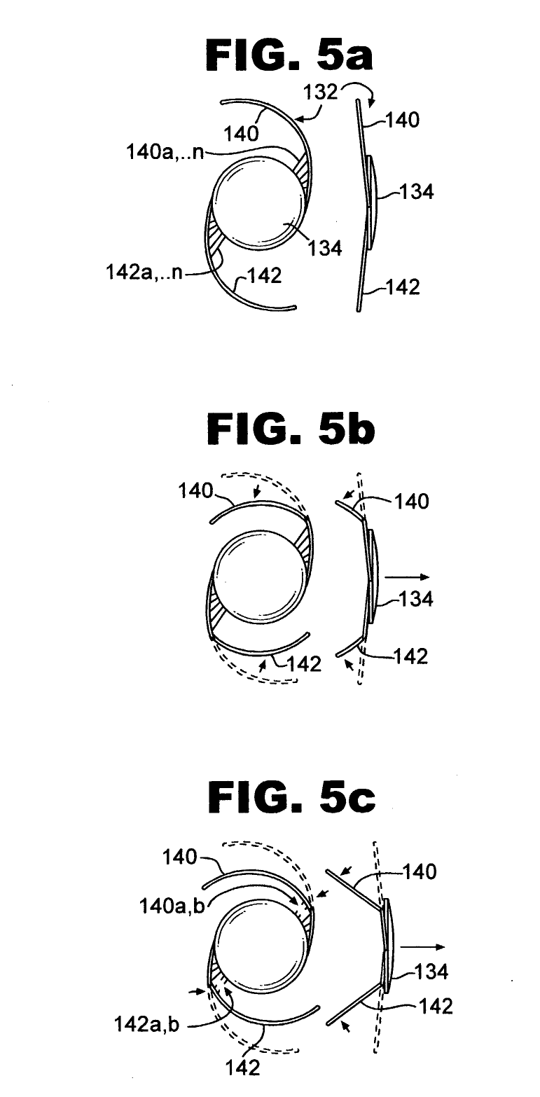

More particularly, a preferred embodiment of the inventive IOL is shown in FIGS. 3 and 4 by the reference numeral 32 and is seen to include an optic 34 having opposite anterior and posterior surfaces 36, 38, respectively, surrounded by a peripheral edge 39. A pair of resilient haptics 40,42 attach to and extend outwardly in a radially curved manner from peripheral edge 38 and are adapted to provide a biasing force in relation to optic 34 to correctly position the IOL within the capsular bag 16. In the preferred embodiment, the haptics 40,42 cause the optic 34 to vault posteriorly toward the retina such that the posterior optic surface 38 presses against the posterior capsular wall 16a. In this regard, it is noted that the posterior peripheral edge of the IOL optic may optionally be provided with a sharp edge (not shown) to inhibit posterior capsular opacification, or secondary cataract.

Referring particularly to FIG. 3, it is seen that at least one, but preferably a plurality of frangible structures in the form of strut elements 40a,b,c and 42a,b,c are formed between optic peripheral edge 39 and respective haptics 40 and 42. Each strut member provides an additional, built-in stress factor to its respective haptic in the inward, radial direction. Should a strut member be removed or severed, the stress factor attributable to that particular strut member is released whereby the resilient force of the corresponding haptic is changed accordingly. It will therefore be appreciated that a surgeon may adjust the resilient force of the haptics by severing one or more strut elements which, in turn, will adjust the degree of optic vault within the capsular bag. The surgeon may quickly and easily sever a strut member in-vivo using a pulsed laser or surgical ophthalmic cutting instrument, for example.

It is noted that frangible structures other than the strut elements illustrated herein may be used to achieve the desired effect of changing the degree of optic vault through severing of at least a portion of the structure. For example, frangible mesh or solid structures formed between the haptic and optic may be used.

Referring to FIGS. 5a,b and c, a preferred embodiment of the inventive IOL is seen in various stages of compression and in-vivo adjustment. More particularly, IOL 132 is seen in FIG. 5a in the uncompressed condition, prior to implantation into an eye. In FIG. 5b, IOL 132 is seen moved from the uncompressed condition (dashed lines), to a compressed condition with haptics 140,142 compressed radially inwardly toward optic 134 (solid lines) upon implantation into an eye whereupon optic 134 vaults in the posterior direction toward the retina. Should this position of the IOL not achieve the desired power correction, the surgeon may sever one or more strut members 140a,b and 142a,b as seen in FIG. 5c whereupon optic 134 moves a bit further in the posterior direction. When designing the IOL, the number of strut members which should be severed is correlated with the degree of optic movement expected and this information would be available to the surgeon. This correlation is easily determined using the IOL design in artificial eye models as well as clinical trial data. In addition to the correlation data, the surgeon may also use the patient's own feedback when making the in-vivo adjustments to IOL 132.

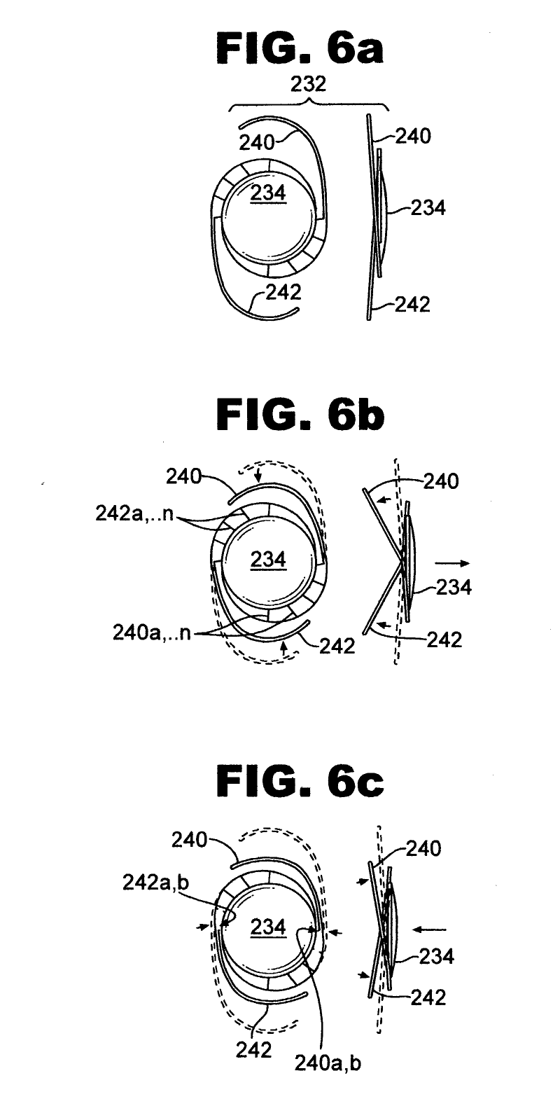

An alternate IOL design is illustrated in FIGS. 6a-c where IOL 234 having haptics 240,242 are interconnected to optic 234 via strut members 240a, . . . n and 242a, . . . n which are more broadly spaced than the strut members of the previously described embodiments. In this embodiment, upon implantation within an eye, the optic vaults posteriorly as seen in FIG. 6b. Assuming this position over-compenstates the needed power correction, the surgeon severs one or more strut members 240a,b and 242a,b whereupon optic 234 vaults in the opposite anterior direction, toward the cornea as seen in FIG. 6c. it will thus be appreciated that the IOL may be designed to move in either the posterior or anterior directions upon severing of one or more of the strut members. In this regard, it is noted that the amount of stress built in to a particular IOL via the strut members may be varied as desired to suit intended applications. Also, the strut members may be tensioned as manufactured, or designed so that the surgeon may him/herself attach strut members to an IOL to achieve a custom IOL having their desired degree of built-in stress. The present invention is applicable to foldable IOLs as well as PMMA IOLs as well as composites thereof.

What is claimed is:

1. An intraocular lens for implanting in a human eye, comprising:
a) a lens optic having opposite anterior and posterior surfaces and a peripheral edge; and
b) at least one haptic attached to and extending from said peripheral edge, said at least one haptic applying a biasing force against said optic to position said optic within said human eye; and
c) a plurality of strut elements extending between said at least one haptic and said peripheral edge, said franglible strut elements configured to provide a predetermined optic power when said intraocular lens is implanted in said human eye, said predetermined optic power being selectively changeable in-vivo by severing one or more of said frangible strut elements.
